(19) United States
(12) Patent Application Publication
(10) Pub. No.: US 2009/0131766 A1
(43) Pub. Date: May 21, 2009

(54) METHOD FOR MONITORING A PATIENT AND SYSTEM FOR IMPLEMENTING SAID METHOD

(76) Inventors: Alexandros Arzimanoglou, Paris (FR); Philippe Ryvlin, Lyon (FR)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(21) Appl. No.: 12/282,995

(22) PCT Filed: Mar. 15, 2007
(86) PCT No.: PCT/EP07/52476

§ 371 Date: Sep. 15, 2008

(30) Foreign Application Priority Data
Mar. 15, 2006 (FR) 06/02272

(51) Int. Cl. A61B 5/00

(52) U.S. Cl. 600/301; 600/300

(57) ABSTRACT

The invention concerns a method for monitoring a patient suffering, for example, from a nervous disease such as epilepsy, characterized in that it consists at least in the following steps: recording continuously video images of the patient in a buffer, determining the occurrence of a seizure of the patient at a time t₀, extracting the video images recorded in the buffer between time t₀−n and t₀+m minutes, and recording the video sequence between t₀−n and t₀+m on a medium. The invention also concerns a method for diagnosing a neurological disease of a patient characterized in that it consists in monitoring the patient using the inventive monitoring method and in viewing the recorded sequence on a display device. Finally, the invention concerns a system for monitoring a patient for implementing said method.

The present invention relates to a method for monitoring a patient and particularly a patient affected by a neurological disease such as epilepsy, and a system for implementing said method.

It is well known how to monitor and optionally record the electroencephalogram (EEG) and/or the electric waves produced by the brain of a patient in order to diagnose a neurological disease such as epilepsy for example.

Considering the bulky and expensive equipment required for such monitoring, this monitoring is generally carried out in a hospital for a period of about 10 days.

However, in order to free hospital beds and to allow monitoring of patients over longer periods, so-called out-patient systems for monitoring the electroencephalogram of a patient have already been devised. For example, this is the case of American patent U.S. Pat. No. 5,029,590 which describes a portable system for detecting vital parameters, in particular an electrocardiogram (ECG) and/or an electroencephalogram (EEG).

The recordings of the electrocardiogram and/or of the electroencephalogram are subsequently analyzed by a physician in order to allow diagnose of the disease and/or to adapt the therapeutic treatment to looking after the patient.

This type of system, although allowing beds to be freed in hospitals, has the drawback of only providing a limited number of pieces of information required for specifically diagnosing the disease and/or of adapting the treatment of the patient. Moreover, this type of system requires the intervention of specialized persons in order to replace on the patient the electrodes which regularly become detached.

One of the objects of the invention is to improve the monitoring systems of the prior art so as to allow a more accurate diagnose of the disease and/or a more efficient adaptation of the treatment to be administered to the patient.

For this purpose, and according to the invention, a method is proposed for monitoring a patient susceptible to be affected by a neurological disease, remarkable in that it consists in at least the following steps:
continuous recording of video images of the patient in a buffer memory,
determination of the occurrence of a fit of the patient at instant t₀,
extraction of video images recorded in the buffer memory between the instant t₀−n and the instant t₀+m minutes, and
recording the video sequence between t₀−n and t₀+m on a medium.

The neurological disease more particularly consists in epilepsy, its therapeutic management depending on the form of epilepsy.

The instant t₀ of the occurrence of a fit of the patient is determined manually at a so-called t′₀ instant by said patient or by any other person.

Alternatively, the instant t₀ of the occurrence of a fit of a patient is automatically determined at a so-called t″₀ instant by at least the succession of the following steps:
recording one or more physiological parameters of the patient, and
comparing the values of the physiological parameter of the patient with pre-recorded threshold values, the occurrence of a fit corresponding to instant t″₀ when one of the values of the physiological parameters of the patient is above one of the threshold values.

The value of n is comprised between 10 and 1 minutes and the value of m is comprised between 10 and 1 minutes.

Moreover, the recorded values of the physiological parameters of the patient are processed prior to their comparison with threshold values.

Advantageously, the instant t₀ of each fit occurrence is recorded in order to determine the time frequency of said fits.

Another object of the invention relates to a method for diagnosing a neurological disease of a patient, remarkable in that it consists of monitoring the patient with the monitoring method according to the invention and of viewing the recorded sequence on a viewing screen.

The recorded video sequence may be viewed on any type of viewing means such as the screen of a computer, a television screen, etc., so that the practitioner upon reading the video sequence may establish a diagnose upon observing the behaviour of the patient during the occurrence of a neurological fit.

A last object of the invention relates to a system for monitoring a patient affected by a nerve disease such as epilepsy for example, for implementing the method according to the invention, remarkable in that it includes at least means for shooting video images, optionally accompanied by a sound track, connected to a processing unit including means for continuously recording video images in buffer memory, means for recording video images on a medium, and triggering means connected to the processing unit and able to be actuated when a fit occurs at instant t₀, actuation of the triggering means providing extraction of a sequence of video images, and optionally of sound tracks, continuously recorded in the buffer memory between instant t₀−n and instant t₀+m, on the one hand, and recording this sequence on a medium on the other hand.

The triggering means preferably consist in at least one manual trigger and, more specifically, in one or more push buttons connected to the processing unit.

Moreover, the push button is advantageously firmly attached to a bracelet positioned on the wrist of the patient.

Other advantages and characteristics will become better apparent from the description which follows, given as a non-limiting example, of the method and system according to the invention with reference to the appended drawings wherein:

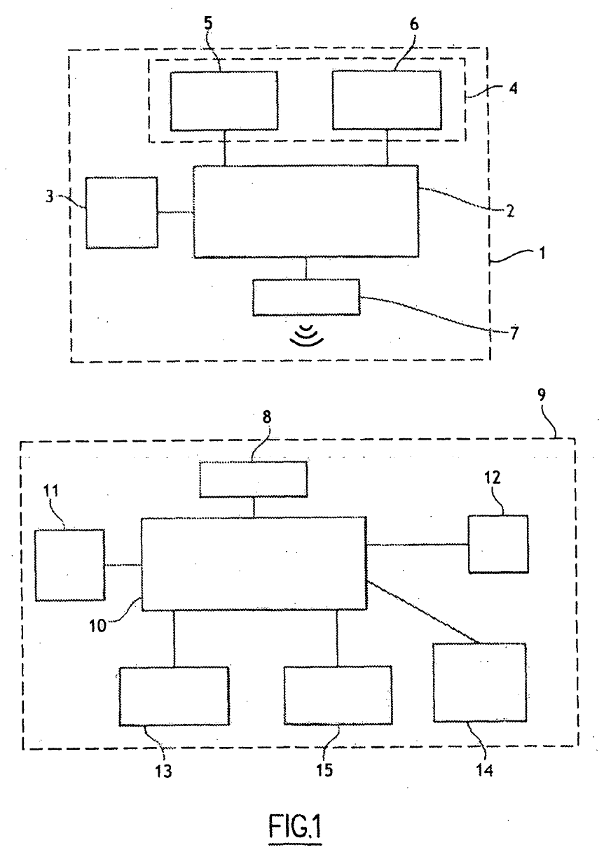

FIG. 1 is a schematic illustration of the system for monitoring a patient according to the invention,

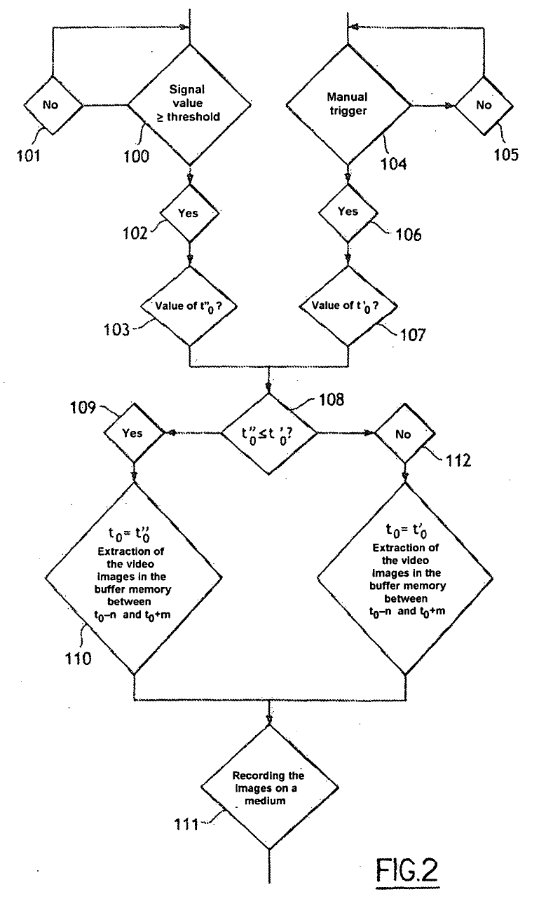

FIG. 2 is a schematic illustration of the flowchart of the computer program for implementing the method according to the invention,

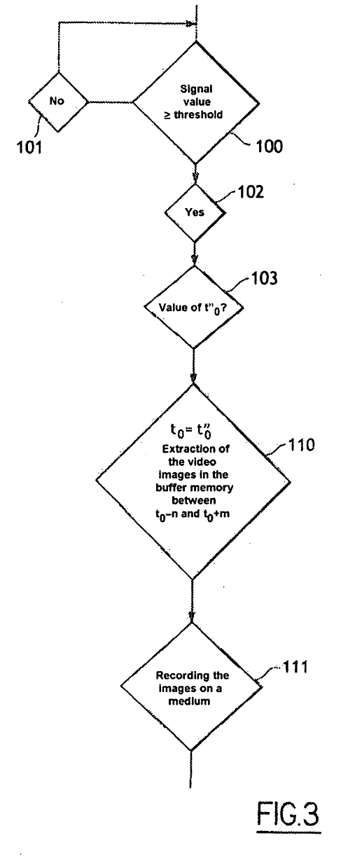

FIG. 3 is a schematic illustration of an alternative embodiment of the flowchart of the computer program for implementing the method according to the invention,

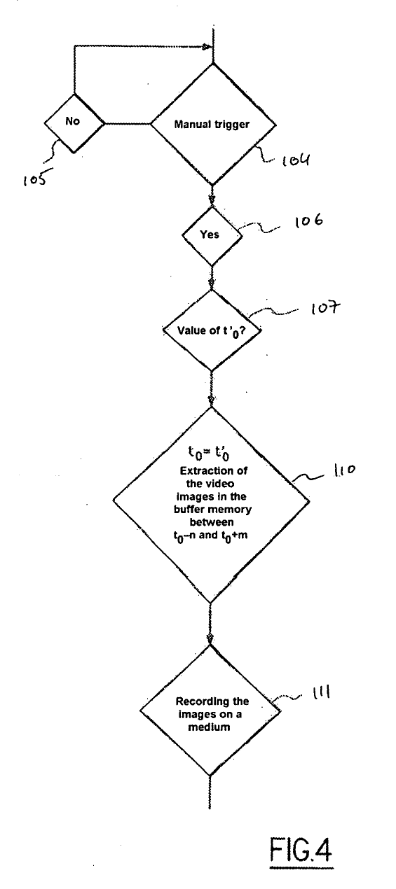

FIG. 4 is a schematic illustration of a second alternative embodiment of the flowchart of the computer program for implementing the method according to the invention,

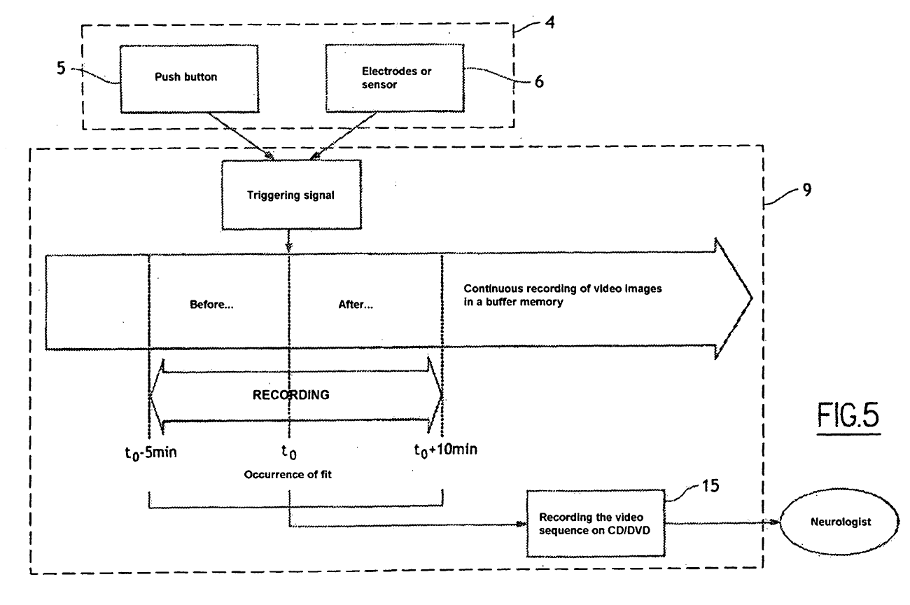

FIG. 5 is a schematic illustration of the operation of the system for monitoring a patient according to the invention.

With reference to FIG. 1, the patient monitoring system according to the invention includes a so-called out-patient unit 1 which may be worn by a patient and comprising a signal acquisition unit 2 powered by a power supply unit 3.

The out-patient unit 1 moreover comprises so-called triggering means 4 connected to the acquisition unit 2 which for example consists in a PC type computer. These triggering means 4 consist in one or more push buttons 5 capable of being actuated by the patient when the latter feels the symptoms of a fit or by a third party, such as a person from the family circle of the patient or a nursing aid, noticing the symptoms of a fit on said patient. When the push button 5 is actuated, the latter transmits a so-called triggering signal to the acquisition unit 2.

A first push button 5 may advantageously be firmly attached to a bracelet positioned on the wrist of the patient, not illustrated in the figures. Said bracelet includes means for transmitting the triggering signal to the acquisition unit 2 which includes corresponding receiving means. These transmitting and receiving means may for example consist in a high frequency radio wave transmitter and receiver or in any other transmitting and receiving means well known to one skilled in the art.

The monitoring system may for example comprise a second push button 5 connected to the acquisition unit 2 and positioned in proximity to the latter as well as a third push button 5 firmly attached to a second bracelet positioned on the wrist of a person from the family circle of the patient. Each push button 5 includes an identification code so that the acquisition unit 2 may recognize the origin of a received signal.

Said triggering means 4 may also consist in electrodes 6 or a multi-modal sensor, capable of being placed on the breast of the patient, on the head or around the wrist of the latter. By “multi-modal” sensor is meant a sensor capable of measuring several quantities.

Thus, these electrodes 6 measure an electrocardiogram (ECG) and transmit these signals to the acquisition unit 2.

Optionally, these electrodes 6 advantageously firmly attached to a helmet put on the head of a patient measure an electroencephalogram (EEG), the signals of which are transmitted to the acquisition unit 2.

Accessorily, the multi-modal sensor may comprise an accelerometer or any equivalent means well known to one skilled in the art for measuring the movements of the patient and allowing triggering as soon as the acquisition unit detects abnormal movements corresponding to the onset of an epilepsy fit.

Said triggering means 4 may advantageously comprise means for cancelling the triggering signal to the acquisition unit 2. This cancelling means may for example consist in two capacitive sensors which, when they are simultaneously pressed for a determined time of the order of a few seconds, transmit a cancelling signal to the acquisition unit. These cancelling means may be positioned on the bracelet bearing the push button 5 or in proximity to the other push buttons 5. These capacitive sensors will advantageously be positioned on either side of the push button 5 on the bracelet. These means may thus also consist in a key of a keyboard for example. Further, the triggering means 4 may advantageously comprise so-called prolongation means for prolonging the recording of the video as this will be detailed later on. These prolongation means may consist in any independent actuating means or in the push button 5.

Moreover, the out-patient unit 1 comprises transmission means 7 connected to the acquisition unit 2 in order to transmit the signals to receiving means 8 of a so-called base unit 9. Said base unit includes a so-called processing unit 10 connected to the receiving means 8. This processing unit 10 for example consists in a PC type computer including a program for processing signals, more particularly signals transmitted by the electrodes as this will be detailed later on. Said processing unit 10 includes an internal clock with which the signals transmitted by the acquisition unit 2 of the out-patient unit 1 may be dated. By the term “dated” is meant the possibility of assigning a time and day to each of the received signals.

Further, the base unit includes a power supply unit 11 powering the processing unit 10 and means for shooting video images 12 such as a video camera for example. This camera will for example consist in a digital camera with infrared sensitivity or a sensitivity of at least 0.2 lux, a resolution of at least 440,000 pixels and a rate of at least 25 images per second.

This base unit 9 also comprises a buffer memory 13 connected to the processing unit 10 or integrated to the latter, in which the video images shot by the camera 12, with the latter oriented towards the patient, are recorded continuously and in a loop.

Moreover, the base unit 9 may advantageously include a viewing screen 14 connected to the processing unit 10 in order to view video images in real time and/or view recorded video images.

Further, the base unit 9 includes recording means 15 connected to the processing unit 10 in order to record video images of the fit of the patient between the instant t₀−n and t₀+m, wherein t₀ is the instant at which the triggering means 6 are actuated, i.e. at an instant corresponding either to the onset of the fit of a patient, or during the fit, or after said fit. These recording means 15 for example consist in a DVD-ROM (Digital Versatile Disc—Read Only Memory) or CD-ROM (Compact Disc—Read Only Memory) burner or else further in a so-called USB (Universal Serial Bus) port capable of receiving a USB key on which video files are recorded. It will be observed that video images of each fit are recorded in a first video file, and information notably relating to the date, the time, the identifier of the triggering means and possibly to the identifier of the transmitter of the signal for prolonging the recording are recorded in a second so-called sub-title file.

It is quite obvious that the DVD-ROM, the CD-ROM or the USB key may be substituted by any other equivalent medium known from the state of the art without however departing from the scope of the invention.

A neurologist, or any other skilled physician, may then view the video images of the fit of a patient recorded on the medium and adapt the treatment depending on his/her observations.

It will be observed that the out-patient unit 1 does not include any recording means nor any video images nor any signals corresponding to the electrocardiogram (ECG) and/or to the electroencephalogram (EEG) so that it is particularly lightweight. At the very most, the out-patient unit 1 includes a buffer memory in which all or part of the signals are recorded temporarily in order to allow them to be transmitted to the base unit 9. Such a lightweight out-patient unit 1 thus allows the patient to move without being bothered by the device for several consecutive days, notably at night.

Moreover, it will be noted that prior to the recording of the video images by recording means on a medium such as a DVD-ROM, a CD-ROM or a USB key, said video images are recorded beforehand either on a buffer memory of the acquisition unit 2 or of the base unit 9 or on the hard disk of said base unit 9.

With reference to FIG. 2, the computer program recorded in the processing unit 10 includes an algorithm comprising a first step 100 for determining an automatic trigger. In this step 100, the signals received by the processing unit 10 are compared in real time with a threshold value recorded in the latter. If the value of the signal received by the processing unit 10 is above said threshold value, in step 101; the algorithm is reinitiated in a loop. On the other hand, if the value of the signal received by the processing unit 10 is below said threshold value, in step 102, the algorithm determines in a step 103, the instant t″₀ at which the value of the signal is below the threshold value from information provided by the internal clock of the processing unit 10.

The signal may correspond to information from the cardiogram (ECG) and/or the electroencephalogram (EEG) and/or to information provided by the multi-modal sensor. The value of the signal then for example corresponds to the cardiac frequency of the patient and/or to the frequency of the brain waves of the patient and/or to the intensity of said waves and/or to the measurement of an abnormal gesture of the patient.

In an alternative embodiment of step 100 for determining an automatic trigger, not illustrated in the figures, the shape of the signals received by the processing unit 10 is compared with one or more shapes of a control signal recorded in the latter. If the shape of the signal received by the processing unit 10 is different from the control signal shape(s), in step 101; the algorithm is reinitiated in a loop. On the other hand, if the shape of the signal received by the processing unit 10 corresponds to the shape(s) of the control signal in step 102, the algorithm determines, in a step 103, the instant t″₀ at which the shape of the signal corresponds to the shape(s) of the control signal from information provided by the internal clock of the processing unit 10.

It will be observed that such an algorithm for recognizing the shape of the signal received by the processing unit has a relatively long execution time so that the instant t₀ corresponds to an instant subsequent to the occurrence of the fit of the patient.

Moreover, the computer program recorded in the processing unit 10 includes an algorithm comprising a step 104 for determining a manual trigger. In this step 104, the algorithm seeks to determine the presence of a triggering signal transmitted by the push button 5 of the system at instant t′₀. If the algorithm does not detect any manual triggering signal, in step 105, either because the system does not comprise any manual triggering means 5 or because the patient does not actuate said manual triggering means, during a sleeping phase for example, said algorithm assigns by default an infinite value to t′₀ and is reinitiated into a loop. On the other hand, if the algorithm detects a manual triggering signal, in step 106, the algorithm determines in step 107, the instant t′₀ at which the manual detection signal is received by the processing unit 10, from information provided by the internal clock of said processing unit 10.

In a step 108, the algorithm then compares t′₀ and t″₀.

If t″₀ is less than or equal to t′₀, i.e. if t″₀ is prior to t′₀, in step 109, the algorithm considers that to corresponds to t″₀ and extracts the video images recorded in the buffer memory of the processing unit 10 between t₀−n and t₀+m, in a step 110, in which to is equal to t″₀. The thereby extracted video images are then compiled in order to form a video sequence which is recorded as a file, in step 111, on a medium such as a DVD-ROM or a USB key for example by the recording means 15. Advantageously, the algorithm assigns a name to said file comprising the date and time of the fit of the patient.

Moreover, it will be observed that the video file consists of a file header, of a frame, of video data and, advantageously sound data, with the assumption that the camera 12 includes a device for recording sounds.

Further, a clock showing the time and day of the recording of the video sequence is advantageously inlaid in the video images.

If t″₀ is larger than t′₀, i.e. if t″₀ is subsequent to t′₀, in step 112, the algorithm considers that t₀ corresponds to t′₀ and extracts video images recorded in the buffer memory of the processing unit 10 between t₀−n and t₀+m, in a step 113, wherein t₀ is equal to t′₀. The thereby extracted video images are then compiled in order to form a video sequence which is recorded as a file, in a step 111, on a medium.

The value n is comprised between 20 and 2 minutes and preferentially equal to 5 minutes, and the value of m is comprised between 20 and 2 minutes, and preferentially equal to 5 minutes. Advantageously, n and m are equal to 5 minutes in order to limit the size of the video files recorded on the medium.

It will be observed that the values of n and m depend on the type of fit of which the patient is a victim and may be parameterized by the practitioner who will record said values of n and m in the acquisition unit 2. Indeed, certain patients have epileptic fits without losing consciousness so that they are able to press on the push button 5 from the onset of the fit; the values of n and m may then be equal to 2 min. Other patients have epileptic fits with loss of consciousness so that they are unable to press on the push button 5, the latter being then pressed by a person from the family circle of said patient; the values of n and m will then be selected preferably equal to 20 min.

The algorithm will then start in a loop, ready for a new fit of the patient.

If the prolongation means are actuated either by the patient or by a person from the family circle between t₀ and t₀+m, the algorithm extracts the video images recorded in the buffer memory of the processing unit 10 between t₀−n and t₀+2m, in step 113. Of course, the prolongation means may be actuated a second time between t₀ and t₀+2m and the algorithm extracts the video images recorded in the buffer memory of the processing unit 10 between t₀−n and t₀+3m, in step 113, and so forth.

If the cancelling means are either actuated by the patient or by a person from the family circle of the patient between t₀ and t₀+m or, under the assumption that the prolongation means would have been actuated once or several times between t₀ and t₀+xm where x is an integer larger than or equal to 2, the recorded images are deleted and the algorithm restarts in a loop in the first step 100.

According to an alternative embodiment, with reference to FIG. 3, the system according to the invention does not include any manual triggering means 5. The algorithm then includes an algorithm comprising a first step 100 for determining an automatic trigger. In this step 100, the signals received by the processing unit 10 are compared in real time with a threshold value recorded in the latter. In the same way as earlier, if the value of the signal received by the processing unit 10 is above said threshold value, in step 101; the algorithm is reinitiated in a loop. On the other hand, if the value of the signal received by the processing unit 10 is below said threshold value, in step 102, the algorithm determines in a step 103, the instant t″₀ at which the value of the signal is below the threshold value from information provided by the internal clock of the processing unit 10.

Then, the algorithm extracts the video images recorded in the buffer memory of the processing unit 10 between t₀−n and t₀+m in a step 110, in which t₀ is equal to t″₀. The thereby extracted video images are then compiled in order to form a video sequence of the epilepsy fit of the patient which is recorded as a file, in step 111, on a medium.

According to another alternative embodiment, with reference to FIG. 4, the system according to the invention does not include any automatic triggering means but only manual triggering means 5. The algorithm then includes an algorithm comprising a step 104 for determining a manual trigger. In this step 104, the algorithm seeks to determine the presence of a triggering signal transmitted by the push button 5 of the system. If the algorithm does not detect any manual triggering signal, in step 105, because the patient or a person of his/her family circle does not actuate said manual triggering means 5, during a sleeping phase for example, said algorithm is reinitiated in a loop, on the other hand, if the algorithm detects a manual triggering signal in step 106, the algorithm determines in step 107, the instant t′₀ at which the manual detection signal is received by the processing unit 10 from information provided by the internal clock of said processing unit 10.

Next, the algorithm extracts the video images recorded in the buffer memory of the processing unit 10 between t₀−n and t₀+m, in a step 110, in which t₀ is equal to t′₀. The thereby extracted video images are then compiled in order to form a video sequence of the epilepsy fit of the patient which is recorded as a file, in a step 111, on a medium.

In order to cancel recording of the video images in the case of untimely pressure on the push button 5, said recording may be cancelled by exerting continuous pressure for several seconds on said push button 5. A signal for cancelling the recording procedure will then be transmitted to the processing unit 10 which will stop execution of the algorithm.

The video sequence corresponding to an epilepsy fit may be recorded in different ways.

According to a first method for compiling the video sequence, a video sequence of a determined duration comprised between 10 and 1 minute, and preferably equal to 5 minutes, are recorded in a buffer memory with continuous deleting of the (n−3)^{th} video sequence. When the acquisition unit 2 detects a triggering signal, the latter compiles the video sequence being recorded upon triggering with the previous and following sequences in order to form only one video sequence which is then recorded on a medium.

According to a second method for compiling the video sequence, an image is recorded every 1/24^{th} second in a data base of the acquisition unit 2, each image file being associated with a date and time to within one hundredth of a second. When the acquisition unit 2 detects a triggering signal at instant t₀, the latter compiles the whole of the image files recorded in the data base between the instants t₀−n and t₀+m in order to form a video sequence including the time succession of said images, said video sequence being recorded on a medium.

It is obvious that the images may be recorded in the data base of the acquisition unit 2 at any frequency such as every 1/12^{th} of a second for example.

Moreover, it is obvious that other methods for compiling the video sequence, well known to one skilled in the art may be contemplated without however departing from the scope of the invention.

Accessorily, the signals corresponding to the electrocardiogram (ECG) and/or to the electroencephalogram (EEG) may also be continuously recorded in a buffer memory and a sequence of said signals comprised between t₀−n and t₀+m may be recorded in a second file on a medium such as a DVD-ROM, preferably on the same medium as the video files.

The operation of the system according to the invention will now be explained with reference to FIGS. 1 and 5.

When a fit occurs at instant t₀, the triggering means 4, i.e. the push button 5 or the automatic triggering means 6, transmit a so-called triggering signal to the processing unit 10. In the absence of receiving a cancelling signal transmitted by the cancelling means, the processing unit 10 then extracts the video images continuously recorded in the buffer memory 13 between instant t₀−5 and instant t₀+10, and then records the video sequence as a computer video file on a DVD-ROM, a USB key or the like.

Of course, under the assumption that one or several prolongation signals would be transmitted to the acquisition unit 2 between t₀ and t₀+10, the processing unit 10 extracts the video images continuously recorded in the buffer memory 13 between instant t₀−5 and instant t₀+x10, where x is the number of prolongation signals, and then records the video sequence as a computer video file and the information relative to the date and to the identifier of the triggering means notably as a computer so-called sub-title file.

A neurologist would be able to view the video sequence of the fit subsequently either from the base unit 9 or by reading the video file of the fit recorded on the DVD-ROM on another computer.

Finally, it is obvious that the examples which have been given are only particular illustrations and are by no means limiting as to the fields of application of the invention.

1. A method for monitoring a patient susceptible to be affected by a neurological disease including at least the following steps:
continuously recording video images of the patient in a buffer memory,
determining the occurrence of a fit of the patient at instant t₀,
extracting video images recorded in the buffer memory between instant t₀−n and instant t₀+m minutes, and
recording the video sequence between t₀−n and t₀+m on a medium.

2. The method according to the preceding claim, wherein the instant t₀ of the occurrence of a fit of the patient is determined manually at a so-called instant t′₀ by said patient or by any other person.

3. The method according to claim 1, wherein the instant t₀ of the occurrence of a fit of a patient is determined automatically at a so-called instant t″₀ by at least the succession of following steps:
recording one or more physiological parameters of the patient, and
comparing the values of the physiological parameters of the patient with pre-recorded threshold values, the occurrence of a fit corresponding to the instant t″₀ when one of the values of the physiological parameters of the patient is above one of the threshold values.

4. The method according to claim 1, wherein the value of n is comprised between 10 and 1 minutes.

5. The method according to claim 1, wherein the value of m is comprised between 10 and 1 minutes.

6. The method according to claim 3, wherein the recorded values of the physiological parameters of the patient are processed prior to their comparison with threshold values.

7. The method according to claim 1, wherein the instant t₀ of each fit occurrence is recorded in order to determine the time frequency of said fits.

8. A method for diagnosing a neurological disease of a patient comprising the monitoring of the patient by:
continuously recording video images of the patient in a buffer memory,
determining the occurrence of a fit of the patient at instant t₀,
extracting video images recorded in the buffer memory between instant t₀−n and instant t₀+m minutes, and
recording the video sequence between t₀−n and t₀+m on a medium,
and of viewing the recorded sequence on a viewing screen.

9. A system for monitoring a patient affected by a nerve disease such as an epilepsy for example, including at least means for shooting video images (12), optionally accompanied by a sound track, connected to a processing unit (10) including means (15) for continuously recording video images into a buffer memory (13), means for recording video images on a medium and triggering means (4) connected to the processing unit (9) and capable of being actuated when a fit occurs at instant t₀, the actuation of the triggering means (4) providing an extraction of a sequence of video images, and optionally of sound tracks, continuously recorded in the buffer memory (13) between instant t₀−n and instant t₀+m, on the one hand, and a recording of this sequence on a medium on the other hand.

10. The system according to claim 9, wherein the triggering means (4) consist in a manual trigger (5).

11. The system according to claim 10, wherein the triggering means (4) consist in one or more push buttons (5) connected to the processing unit (10).

12. The system according to claim 11, wherein the push button (5) is firmly attached to a bracelet positioned on the wrist of the patient.

13. The system according to claim 11, wherein the push button (5) is firmly attached to a bracelet positioned on the wrist of a person from the family circle of the patient.

14. The system according to claim 11, wherein the push button (5) is positioned in proximity to the acquisition unit (2).

15. The system according to claim 9, wherein the triggering means (4) consist in at least one sensor (6) connected to the processing unit (10) and capable of reading one or more physiological parameters of the patient.

16. The system according to claim 9 further including a base unit (9) including the processing unit (10), the video image shooting means (12) and optionally audio shooting means, connected to the processing unit (10) and means (8) for receiving signals on the one hand, and a so-called out-patient unit (1) capable of being worn by the patient and including triggering means (4), a unit (2) for acquiring signals and a unit (7) for transmitting said signals cooperating with the receiving unit (8) connected to the processing unit (9) on the other hand.

17. The system according to claim 9 wherein it includes a viewing screen (14) connected to the processing unit (9).
